(19) United States
(12) Patent Application Publication
(10) Pub. No.: US 2026/0335710 A1
(43) Pub. Date: Oct. 29, 2026

(54) METHOD FOR AUTOMATED EXECUTION OF HYPOXIA TRAINING

(71) Applicant: Egor EGOROV, Berlin (DE)

(72) Inventor: Egor EGOROV, Berlin (DE)

(21) Appl. No.: 19/102,471

(22) PCT Filed: Aug. 11, 2023
(86) PCT No.: PCT/EP2023/072298

§ 371 Date: Feb. 10, 2025

(30) Foreign Application Priority Data
Aug. 12, 2022 (DE) 10 2022 120 403.0

(51) Int. Cl. A63B 23/18; A63B 24/00; G16H 20/30

(52) U.S. Cl. CPC A63B 23/18; A63B 24/0075; G16H 20/30; A63B 2214/00; A63B 2230/436

(57) ABSTRACT

The invention relates to a method for automated execution of a hypoxia training with the method steps of: acquiring a first measured value or inputting a first parameter, acquiring a second measured value or inputting a second parameter, determining training parameters from the first acquired measured value and/or the first input parameter and the second acquired measured value and/or the second input parameter, creating a hypoxia training program and/or executing a hypoxia training with the determined training parameters, wherein the training parameters comprise one or more parameters from the group duration of a hypoxia interval, number of cycles of the hypoxia intervals and minimum oxygen content of a breathing air mixture provided during the hypoxia training, and wherein at least one value of one of the two training parameters is at least 1% and at most 25% above or below the value of that training parameter which could be determined solely from the first acquired measured value and/or the first input parameter, or solely from the second acquired measured value and/or the second input parameter.

The invention relates to a method for automated execution of hypoxia training with the method steps of: acquiring a first measured value or inputting a first parameter, acquiring a second measured value or inputting a second parameter, determining training parameters from the first acquired measured value and/or the first input parameter and the second acquired measured value and/or the second input parameter, creating a hypoxia training program and/or executing a hypoxia training using the determined training parameters, wherein the training parameters comprise one or more parameters from the group consisting of duration of a hypoxia interval, number of cycles of the hypoxia intervals and minimum oxygen content of a breathing air mixture provided during the hypoxia training, and wherein at least one value of one of the two training parameters is at least 1% and at most 25% above or below the value of that training parameter which could be determined solely from the first acquired measured value and/or the first input parameter, or solely from the second acquired measured value and/or the second input parameter.

### PRIOR ART

Hypoxia can cause reactions in any cell in the body and can enable an increased energy metabolism. It can contribute to the activation of a variety of genes. Athletes, healthy people, and the sick may benefit from hypoxia.

It has been known for a long time that altitude training has benefitial effects. But how the slight lack of oxygen leads to an improvement in performance in the body could not be explained sufficiently until a few years ago. The observed increase in red blood cells was not sufficient to explain the changes in the body. The breakthrough for understanding came with the discovery of the hypoxia-inducible factor HIF-1-alpha. It provided the explanation for the comprehensive effect of altitude training. Abbreviation HIF stands for Hypoxia-Inducible Factor. Behind the technical term lies an oxygen sensor, which becomes active when there is not enough oxygen in the body cells. It controls one of the most important processes in the body for survival: the adaptation of cells, tissues and organs to a lack of oxygen. At the same time, it is a signal for self-repair in the body.

The best known positive effect of HIF is erythropoietin synthesis (EPO) in the kidney and liver. It served to explain the changes in the cardiovascular, respiratory and blood systems before the discovery of HIF. It is now clear that performance improvement is much more comprehensive. The endothelial cells of the tunica intima react to the impact of hypoxia with an increased nitric oxide synthesis (NO). The gas has a decisive impact on the dilatation of the vessels. It leaves the endothelium and has a relaxing effect on smooth muscle cells in the surrounding tissue. On the intimal tunica itself, NO prevents platelet adhesion and aggregation. It is also interesting in this context that the endothelial cells form the vasculating factor VEGF under the impact of hypoxia. As a result of its formation, neoangiogenesis of the capillary vessels occurs in the context of hypoxia therapy. Very often, these additional vessels are located in damaged or poorly perfused tissue sections.

The heart beats faster and people breathe more often. The body tries to absorb as much oxygen as possible from the air it breathes. For humans, this is neither unpleasant nor dangerous. The vessels widen, and blood can flow faster. The number of red blood cells is elevated, and the body can absorb more oxygen molecules. Supply to all organs is improved. In addition, the body starts an adjustment program. Under the impact of hypoxia factor HIF-1-alpha, the body takes all precautions to get by even with less oxygen.

The mitochondria become dense and rejuvenated. The energy production is optimized, which leads to more physical and mental performance in everyday life. In people with disorders, symptoms lessen. In the brain, the transmission of nerve impulses improves. In some areas, new nerve cells develop. Consquently, mental strength increases and the risk of dementia and Parkinson's disease is lowered. The vascular network becomes denser. The inner walls of the vessels become smooth and supple. This reduces the chance of dangerous blood clots, as well as the risk of heart attack and stroke.

On the other hand, lack of oxygen can lead to inability to concentrate, fatigue, and eventually loss of consciousness, causing significant damage. An optimal training program must therefore take into account the personal limits of each person to be trained in order to be safe for the subject, but should also be in an area in which the organism can be optimally addressed and the training is effective.

It is therefore an object of the present invention to provide a method for automated execution of hypoxia training, which enables a safe and at the same time efficient execution of hypoxia training. Furthermore, it is an object of the invention to provide training parameters for different parameters which ensure a safe and at the same time efficient execution of hypoxia training.

The object mentioned above is achieved by means of the method for the automated execution of a hypoxia training according to claim 1. Further advantageous configurations of the invention are set out in the dependent claims.

The method according to the invention for the automated execution of a hypoxia training has four method steps: In the first method step, a first measured value is acquired or a first parameter is input. In the second method step, a second measured value is acquired or a second parameter is input. The first and/or second method steps are usually executed under medical supervision as part of an initial test on a user. The initial test objectively determines the sensitivity of the user's body to the hypoxic stimulus. The initial test is used to adjust the intensity of hypoxia training to the user's needs. The acquired measured values make it possible to detect outliers and incorrect measurements which, when selecting the determination of the training parameters, can otherwise cause possible damage to the patient during hypoxia training.

In the third method step, training parameters are determined from the first acquired measured value and/or the first input parameter and the second acquired measured value and/or the second input parameter.

In the fourth method step, a hypoxia training program is created and/or a hypoxia training is executed with the specific training parameters. The training parameters comprise one or more parameters from the group duration of a hypoxia interval, number of cycles of the hypoxia intervals, minimum oxygen content of a breathing air mixture provided during the hypoxia training, and/or length of the breaks between the hypoxia intervals. The training program is created automatically, i.e. the measured values and entered parameters are automated converted into a training program.

The hypoxia training is optimally adjusted to the user's needs via the setting options for the training parameters duration, number of cycles, oxygen content and oxygen saturation in the blood. Based on the acquired measured values and the input parameters, the oxygen reduction in the breathing air achieves the highest effectiveness and the greatest safety during training.

These training parameters are selected in such a way that all adaptation processes in the body are triggered, but no damage can be caused by the intentionally induced oxygen deficiency. For this purpose, at least one value of one of the two training parameters is at least 1% and at most 25% above or below the value of that training parameter which could be determined solely from the first acquired measured value and/or the first input parameter, or solely from the second acquired measured value and/or the second input parameter. The training parameter is here towards facilitated training conditions in relation to the training parameter determined solely from the first acquired measured value and/or the first input parameter, or solely from the second acquired measured value and/or the second input value, that is to say 1% to 25% above the target value of the oxygen saturation in the blood, above the oxygen content in the breathing gas, below the length of the hypoxia interval, above the length of the pause between the hypoxia intervals, below the number of repetitions. Preferably, the training parameter is the parameter with the facilitated training conditions solely from the first acquired measured value and/or the first input parameter or solely from the second acquired measured value and/or the second input parameter, that is to say 1% to 25% above the target value of the oxygen saturation in the blood, above the oxygen content in the breathing gas, below the length of the hypoxia interval, above the length of the pause between the hypoxia intervals, below the number of repetitions. This tolerance of the parameter deviation ensures safe carrying out of the hypoxia training program for the user, while at the same time achieving a high training effect. Many tests have shown that a training program whose training parameters are in this area avoids an oxygen undersupply which damages the organism, but is nevertheless in an area in which the training is effective. In a preferred embodiment of the invention, at least one value of one of the two training parameters is at least 1% and at most 20%, preferably at least 1% and at most 15%, and particularly preferably at least 1% and at most 10%, above or below the value of that training parameter which could be determined solely from the first acquired measured value and/or the first input parameter or solely from the second acquired measured value and/or the second input parameter.

In a refinement of the invention, the value of the one training parameter is at least 1.5% and at most 8% above or below the value of the training parameter. In a further aspect of the invention, the value of the one training parameter is at least 2% and at most 7% above or below the value of the training parameter. Further tolerances are conceivable, depending on the acquired measured values of the user, for example at least 3% and at most 7.5%, at least 4% and at most 7%. The tolerances are chosen as small as possible in order to achieve an effective hypoxia training, but at the same time large enough to make the hypoxia training safe for the user.

In a further embodiment of the invention, the value of one training parameter is set independently of the results of the acquired measured values and/or input parameters. The further training parameter is adapted accordingly. Thus, only one training parameter is varied, while a further training parameter is set as a function of the measured values and/or input parameters and is not variable.

In a further embodiment of the invention, the first and second process steps take place at different points in time, preferably at least 10 minutes apart, more preferably at least 30 minutes apart, and even more preferably at least 24 hours apart. By shifting the points in time, incorrect measurements can be uncovered, for example, those due to physical deviations from normal and/or physical irregularities.

In a further embodiment of the invention, the first and/or second process steps and third and/or fourth process steps take place at different points in time, preferably at least 10 minutes apart, more preferably at least 30 minutes apart, and even more preferably at least 24 hours apart. The determination of the training parameters and/or the creation and/or execution of the hypoxia training program cannot be compared with a monitoring and adaptation of a hypoxia training program already created during the execution of a hypoxia training program.

In a further configuration of the invention, the training parameters are determined anew. The training parameters are created independently of previously created training parameters and/or hypoxia training programs already executed for the same subject.

In a further configuration of the invention, the at least one training parameter is the duration of a hypoxia interval and/or the minimum oxygen content of a breathing air mixture provided during the hypoxia training. By means of these two training parameters, hypoxia training is tailored to the needs of the user. These training parameters are determined in such a way that all adaptation processes in the body are triggered, but no damage is caused by the intentionally induced oxygen deficiency.

In an advantageous embodiment of the invention, the hypoxia training program is created automatically. This makes the hypoxia training program reproducible, human error sources are minimized and the hypoxia training program can be created cost-efficiently.

In a further advantageous configuration of the invention, the hypoxia training program is executed automatically. The hypoxia training program can therefore be executed cost-effectively for the user, in particular if the user is wearing a monitoring device, for example a pulse oximeter.

In a further embodiment of the invention, the measured values and/or input parameters comprise the SI value of a heart rate variability measurement, the time of a breath hold test, the results of a MitoOx test, the results of an MDA-LDL test, the dose determination according to Prof. Apanasenko, the results from tests for basal respiration, ATP production, proton leak, maximum respiration, reserve capacity and/or non-mitochondrial respiration.

Heart rate variability can be used to measure a patient's physical resilience. Measurement is recommended before and during hypoxia training.

Heart rate variability (HRV) can be used to measure the general adaptability of the organism. The measurement provides an assessment of how well the autonomic nervous system can respond to stressful influences via its two components, the sympathetic and parasympathetic nervous system. HRV values are of interest for hypoxia training because they provide information on the ability of the autonomic nervous system to regulate. The flexibility of the autonomic nervous system is important when planning the duration of the interval. During the course of the training, they can be used for control.

Various HRV parameters are included in the SI value. The magnitude of the SI can be used to estimate how balanced the activity of the sympathetic and parasympathetic nervous systems is. Higher SI values are an indication of a tightness of the regulation system and of a lower relaxation capacity.

The result of the breath hold test provides first indications of the possible depth of hypoxia. It also provides information on load capacity during training. There are different breath hold tests, the Stange and the Genchi tests are just two of them. They provide the same information and differ only in the approach, which attracts the attention of different patient groups. Both tests are executed before the first hypoxia training and after completion of the hypoxia course of treatment. A test result with longer breath holding times is confirmation of the correct dosage and of the positive effect of the hypoxia.

Whenever possible, mitochondrial pool analysis should be performed in all patients. The assessment of mitochondrial function was only indirectly possible for a long time and associated with high costs for the patients. A new approach is to evaluate mitochondrial DNA (mtDNA) from a drop of blood by means of the MitoOx test. Any change, from a tiny point mutation to the loss of larger sections of mtDNA (deletions), affects the mitochondrial respiratory chain. The MitoOx test provides a determination of the most common deletion 4977 bp (common deletion). It comprises base pairs 8470 to 13447 and affects about one third of the total mitochondrial genome. Complexes I, IV, and V of the respiratory chain are affected by the mutation. The number of deletions 4977 bp increases with the oxidative load.

The MitoOx test compares the number of healthy mitochondrial gene copies to the number of damaged gene copies. The higher the number of healthy mitochondrial gene copies, the lower the oxidative load or the higher the antioxidative capacity. If the proportion of copies with the deletion 4977 bp increases, it can be assumed that mitochondrial function is reduced.

For hypoxia training, this means that a hyperoxia phase is counterproductive in patients with an accumulation of deletion 4977 bp. The oxidative stress is further elevated by the elevated oxygen content. In patients with mitochondrial dysfunction, switching between a hypoxia and a hyperoxia phase may cause further deterioration of the mitochondria.

Most chronic diseases are associated with mitochondrial dysfunction. The Bioenergetic Health Index (BHI) examines the functioning of the individual switching cells in the respiratory chain and assesses the bioenergetic quality of the mitochondria. Six parameters can be used to identify weak points and determine the extent of dysfunction or mitochondrial efficiency. Other acquired measured values and/or input parameters include results from tests of basal respiration, ATP production, proton leak, maximum respiration, reserve capacity, and/or non-mitochondrial respiration.

In a further embodiment of the invention, the training parameters are checked before the training program is created. During the check, the training parameters are compared with the values measured in real time, e.g. for oxygen concentration in the breathing gas, oxygen saturation of the blood and the heart rate of the user.

In a further embodiment of the invention, the training parameters are changed before the training program is created. If the determined training parameters are within the range anticipated during the initial test, a training program is created and the training plan is started with the determined training parameters. If the determined training parameters are not within the range anticipated during the initial test, the training parameters are changed in such a way that measured values for, for example, oxygen concentration in the breathing gas, oxygen saturation of the blood and heart rate will be again in the anticipated range. A new training plan with the changed training parameters is then created and started with the changed training parameters. By checking the training parameters and, if necessary, changing the training parameters in real time, safe and at the same time effective hypoxia training is achieved.

In a refinement of the invention, the training program created is created on a first computer unit. In particular, the training program created is created under medical supervision. This makes the hypoxia training program reproducible, human error sources are minimized and the hypoxia training program can be created cost-efficiently.

In a further embodiment of the invention, the training program created is transmitted from a first computer unit to a second computer unit. The second computer unit is usually arranged remotely from the first computer unit.

In a further configuration of the invention, the second computer unit includes a controller for executing the training program. With the second computer unit, the hypoxia training program created can also be executed remotely from the first computer unit. A user can execute the hypoxia training program created, for example, in his home, by means of the second computer unit and the controller arranged therein or connected to the second computer unit, in order to execute the training program.

In a further configuration of the invention, the training parameters comprise the number of hypoxia cycles, the number of trainings of the course of treatment and/or the regeneration time between the individual trainings. The intensity of the training is largely determined by the number of hypoxia intervals. 4-5 cycles per training session are sufficient according to scientific findings. Patients with poor baseline values begin hypoxia training with 3 to 4 cycles. After about five training sessions, the training curves are compared. Changes in HRV parameters, pulse and oxygen saturation curves are expected. For patients in a reduced general condition, intermediate tests, such as an HRV measurement or a breath hold test, are also useful for evaluating the training.

A further embodiment comprises regeneration phases in the form of weekly application repetitions. In particular, the repetitions increase with higher target values and/or higher training parameters. This prevents heavy physical loads and allows physical adaptations during the regeneration phases.

After a number of training units, preferably after a maximum of 5 training units, even more preferably after a maximum of 2-4 training units, most preferably after each training unit, the training parameters are determined anew. In this way, an effective and at the same time safe personalized hypoxia training can be ensured.

In a further aspect of the invention, the method is executed in a personalized manner. Each user reacts individually to the hypoxia training. Intermediate tests, such as heart rate variability (HRV) measurement, should be done periodically, especially in patients in a reduced general condition, to avoid overtraining. In addition, there are individual factors such as a tendency to hypoglycaemia and the use of medications, which are included in the training planning.

In a further embodiment of the invention, the training program is discontinued as a function of combinations of two values from the acquired measured values and/or input parameters and/or as a function of a single value of the acquired measured values and/or input parameters. In particular, a safety limit in the form of parameters which lead to the discontinuation of the automated training and/or the creation of the automated training (safety cut-off) is set for the oxygen saturation of the blood individually for a user. If this safety limit is not reached, a safety program is activated in such a way that the oxygen saturation of the blood rises again above the safety limit. This makes a safe training program possible for the user even outside of medical supervision.

In a refinement of the invention, the automatic creation and/or execution of the hypoxia training is discontinued if the training parameters determined from the first acquired measured value and/or the first input parameter or only from the second acquired measured value and/or the second input parameter exceed the tolerance of the parameter deviation. In this way, it can be ensured that an oxygen deficiency which damages the organism is avoided.

In one embodiment, the hypoxia training comprises only hypoxia phases and hyperxia phases in order to effect particularly high performance increases.

In another embodiment, the hypoxia training comprises only hypoxia phases and normoxia phases in order to reduce the load on the body. Hyperoxia phases in particular can cause high loads for users.

In a further embodiment, the hypoxia training comprises hyperoxia and normoxia phases after each hypoxia phase in order to reduce load peaks of the body, especially towards the end of the training, but nevertheless to enable an elevated increase in performance. The training preferably begins with a hyperoxia phase and/or hyperoxia and normoxia phases are carried out alternately in each case after hypoxia phases. Especially the change between hyperoxia and normoxia phases reduces the load peaks within the hypoxia training.

Exemplary embodiments of the method according to the invention for automated execution of an hypoxy training are shown schematically in the drawings in a simplified manner and are explained in more detail in the following description.

In the drawings:

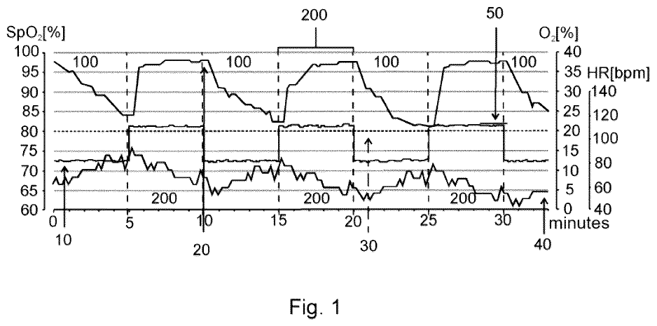

FIG. 1: shows a diagram of a hypoxia-normoxia training plan

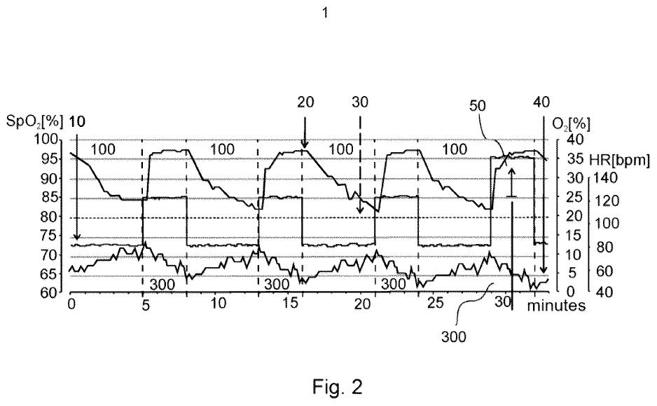

FIG. 2: shows a diagram of a hypoxia-hyperoxia training plan

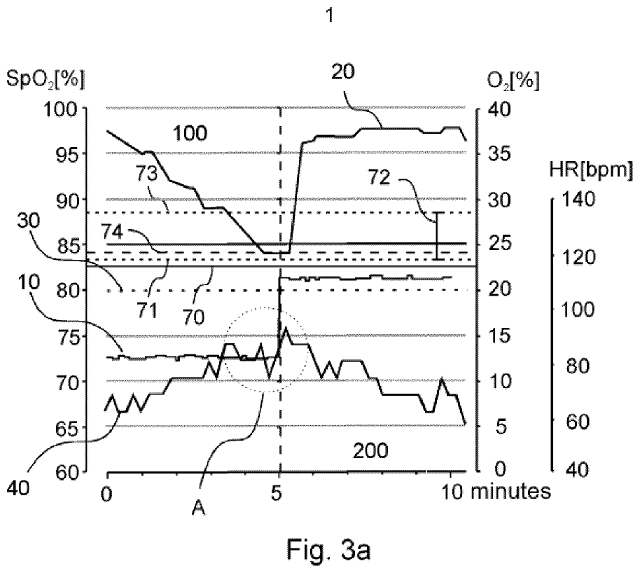

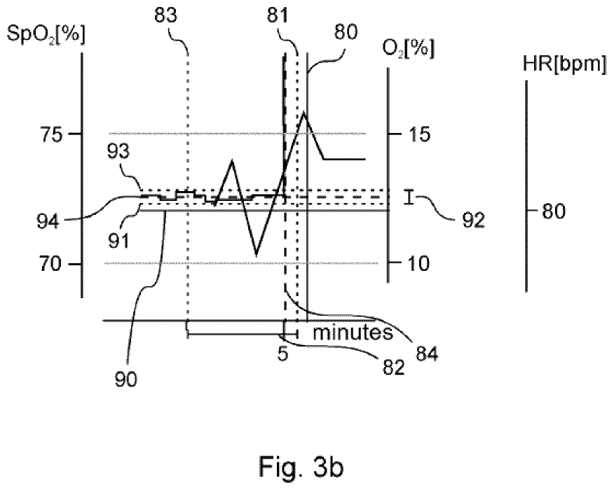

FIG. 3a: shows a detailed view of a hypoxia-normoxia training plan

FIG. 3b: shows a detailed view of a hypoxia-normoxia training plan with tolerances of the training parameters marked

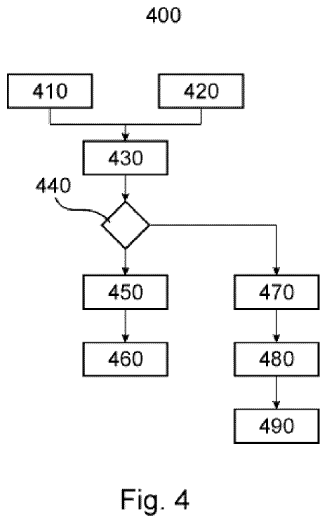

FIG. 4: shows a flow chart of the method according to the invention for the automated execution of a hypoxia training

FIG. 1 shows an exemplary embodiment of a hypoxia-normoxia training program 1 which is executed at intervals. Hypoxia intervals 100 alternate with normoxia intervals 200. Each interval 100, 200 in this exemplary embodiment has a duration of 5 minutes. The diagram shows the values for oxygen concentration in the breathing gas 10 (ordinate O2), oxygen saturation of blood 20 (ordinate SpO2), the heart rate 40 (ordinate HR) and a safety limit 30 (safety cut-off) for oxygen saturation of blood 20 SpO2-over time (abscissa). The safety limit 30 is 80% SpO2 in this and all subsequent exemplary embodiments, but can be set individually for each user. If this safety limit 30 is not reached, a safety program is activated in such a way that oxygen saturation of blood 20 rises again above the safety limit 30 of 80% SPO2.

For executing method 400 according to the invention for the automated execution of a hypoxia training 400 (cf. FIG. 4), a plurality of measured values of a user are acquired 410 by means of an initial test and a plurality of parameters of a user are input 420 into a first computer unit.

Heart rate variability can be used to measure a patient's physical resilience. Measurement is recommended before and during hypoxia training. Heart rate variability (HRV) can be used to measure the general adaptability of the organism. The measurement provides an assessment of how well the autonomic nervous system can respond to stressful influences via its two components, the sympathetic and parasympathetic nervous system. HRV values are of interest for hypoxia training because they provide information on the ability of the autonomic nervous system to regulate. The flexibility of the autonomic nervous system is important when planning the duration of the interval. During the course of the training, they can be used for control.

Various HRV parameters are included in the SI value. The magnitude of the SI can be used to estimate how balanced the activity of the sympathetic and parasympathetic nervous systems is. Higher SI values are an indication of a tightness of the regulation system and of a lower relaxation capacity.

The result of the breath hold test provides first indications of the possible depth of hypoxia. It also provides information on load capacity during training. There are different breath hold tests, the Stange and the Genchi tests are just two of them. They provide the same information and differ only in the approach, which attracts the attention of different patient groups. Both tests are executed before the first hypoxia training and after completion of the hypoxia course of treatment. A test result with longer breath holding times is confirmation of the correct dosage and of the positive effect of the hypoxia.

Whenever possible, mitochondrial pool analysis should be performed in all patients. The assessment of mitochondrial function was only indirectly possible for a long time and associated with high costs for the patients. A new approach is to evaluate mitochondrial DNA (mtDNA) from a drop of blood by means of the MitoOx test. Any change, from a tiny point mutation to the loss of larger sections of mtDNA (deletions), affects the mitochondrial respiratory chain. The MitoOx test provides a determination of the most common deletion 4977 bp (common deletion). It comprises base pairs 8470 to 13447 and affects about one third of the total mitochondrial genome. Complexes I, IV, and V of the respiratory chain are affected by the mutation. The number of deletions 4977 bp increases with the oxidative load.

The MitoOx test compares the number of healthy mitochondrial gene copies to the number of damaged gene copies. The higher the number of healthy mitochondrial gene copies, the lower the oxidative load or the higher the antioxidative capacity. If the proportion of copies with the deletion 4977 bp increases, it can be assumed that mitochondrial function is reduced.

Most chronic diseases are associated with mitochondrial dysfunction. The Bioenergetic Health Index (BHI) examines the functioning of the individual switching cells in the respiratory chain and assesses the bioenergetic quality of the mitochondria. Six parameters can be used to identify weak points and determine the extent of dysfunction or mitochondrial efficiency. These six parameters are tests of a user's basal respiration, ATP production, proton leak, maximum respiration, reserve capacity and/or non-mitochondrial respiration. ests of basal respiration, ATP production, proton leak, maximum respiration, reserve capacity, and/or non-mitochondrial respiration.

The training parameters are determined 430 from these measured values and input parameters. The training parameters comprise the duration of a hypoxia interval 100, the duration of the normoxia interval 200, the number of cycles of the hypoxia intervals and the minimum oxygen content of a breathing air mixture 10 provided during the hypoxia training. Hypoxia phase 100 is individually adjusted via the setting options of the duration and frequency of the intervals 100, 200, the oxygen reduction of breathing gas 10 and oxygen saturation in blood 20.

These training parameters are selected in a personalized manner for a user in such a way that all adaptation processes in the body are triggered, but no damage can be caused by the intentionally induced oxygen deficiency. Training program 1 thus created is then transmitted to a second computer unit which has a controller for executing training program 1. Subsequently, training program 1 is executed 460 on a suitable training device.

For example, if values of 40/45 seconds are reached during the breath hold test according to Stange/Genchi, values of MitoOx>10⁸:1 during laboratory testing (MitoOx test, MDA-LDL), MDA-LDL normal to slightly elevated, BHI>2.0, values of >15 points for both parameters during dose determination according to Professor Apanasenko, hypoxia training can be executed with training parameters with respect to target values of 80-82 SpO2, the duration of the hypoxia phase being at least 5 minutes, having 6 cycles, can take place up to 3 times a week and comprising 10-14 training units per course of treatment.

For example, if values of 25-40/30-45 seconds are reached during the breath hold test according to Stange/Genchi, values of MitoOx 10⁸:1 during laboratory testing (MitoOx test, MDA-LDL), MDA-LDL slightly elevated, BHI 2.0-1.5, values of 10-15 points for both parameters during dose determination according to Professor Apanasenko, hypoxia training can be executed with training parameters with respect to target values of 83-84 SpO2, the duration of the hypoxia phase being at least 4 minutes, having 5 cycles, can take place up to 3 times a week and comprising 15-20 training units per course of treatment.

For example, if values of 10-24/15-29 seconds are reached during the breath hold test according to Stange/Genchi, values of MitoOx 10⁷ to >10⁵:1 during laboratory testing (MitoOx test, MDA-LDL), MDA-LDL slightly to significantly elevated, BHI <1.5, values of 4-6 points for both parameters during dose determination according to Professor Apanasenko, hypoxia training can be executed with training parameters with respect to target values of 85-87 SpO2, the duration of the hypoxia phase being at least 3 minutes, having 4 cycles, can take place up to 3 times a week and comprising 15-20 training units per course of treatment.

In one embodiment, the parameters from different tests can be combined with one another in order to determine the training parameters.

For example, if during the breath hold test according to Stange/Genchi values are reached over 40/45 seconds for one parameter and a value between 25-40/30-45 seconds, the one parameter comprises a training parameter with respect to target values of 80-82 SpO2 and the other parameter comprises a training parameter with respect to target values of 83-85. The values do not exceed the tolerance of the deviation parameters, but in order to ensure the safety of the user, the training parameters for the smaller target value are used as the basis for hypoxia training, the duration of the hypoxia phase being 4-5 minutes, having 5 cycles, can take place up to 3 times a week and comprising 15-20 training units per course of treatment.

Training program 1 begins with a hypoxia interval 100, which ends after 5 minutes with an oxygen saturation of blood 20 of 85 percent. An oxygen saturation of blood 20 below 85 percent depends on the user's state of health and fitness as well as on the results of the initial tests.

The curve of heart rate 40 runs counter to the curve of oxygen concentration in breathing gas 10. In the hypoxia phase, heart rate 40 increases and in the normoxia or hyperoxia phase, heart rate 40 decreases again.

Each hypoxia interval 100 is followed by a normoxia interval 200 with oxygen-enriched or normally saturated room air 200. The length of normoxia interval 200 is also 5 min in this exemplary embodiment. For the body, normoxia interval 200 is like a rest. Heart rate 40 slows down again and oxygen saturation of blood 20 rises again up to 99%.

FIG. 2 shows an exemplary embodiment of a hypoxia-hyperoxia training program 1, which is also executed at intervals. Hypoxia intervals 100 alternate with hyperxia intervals 300. Each hypoxia interval 100 in this exemplary embodiment has a duration of 5 minutes.

The diagram, on the other hand, shows the values for oxygen concentration in breathing gas 10 (ordinate O2), oxygen saturation of blood 20 (ordinate SpO2), heart rate 40 (ordinate HR) and a safety limit 30 (safety cut-off) for oxygen saturation of blood 20 SpO2-over time (abscissa).

If hypoxia interval 100 is alternated with a slightly elevated hyperoxia phase 300 (from 25 percent to 35 percent oxygen concentration in breathing gas 10), the values normalize faster than with a normoxia phase 200 (cf. FIG. 1) Because the body can elevate oxygen saturation of blood 20 more rapidly in a hyperoxia phase 300, the duration of hyperoxia phase 300 is shorter than in a normoxia phase 100, in this exemplary embodiment 3 minutes.

Many users estimate the relaxation effect in hyperoxia phase 300 to be higher than in normoxia phase 100. Physiologically, however, hyperoxia phase 300 is just as much a load on the body as hypoxia phase 100, because it triggers another stress stimulus in the body. The elevated oxygen supply in breathing gas 10 leads to greater load and stimulation of the body's own antioxidant systems. The oxidative stress is elevated. Hypoxia-hyperoxia training 1 can therefore be overwhelming for users with health impairments.

Scientific studies and the experience of the past four decades have shown that it is precisely the change between a short-term reduction in oxygen saturation of blood 20 and a normal oxygen saturation of blood 20 that brings the best health effects. In contrast, a longer reduction in oxygen saturation of blood 20, comparable to a longer stay in the mountains or in a hypoxic chamber, would have a lower training effect. The more frequent change leads to an additional stimulation of the adaptability of the body. Comparison of different training protocols resulted in the recommendation of 4-5 cycles with a hypoxia interval of 100 of 5-7 minutes followed by normoxia interval 200 or hyperxia interval 300.

FIGS. 3a and 3b show detailed views of an exemplary embodiment of a hypoxia-normoxia training program 1. FIG. 3 b shows an enlarged section A of the diagram in FIG. 3a. The diagram shown in FIGS. 3a and 3b, on the other hand, shows the values for oxygen concentration in breathing gas 10 (ordinate O2), oxygen saturation of blood 20 (ordinate SpO2), heart rate 40 (ordinate HR) and a safety limit 30 (safety cut-off) for oxygen saturation of blood 20 SpO2-over time (abscissa). Safety limit 30 is 80% SpO2 in this and all subsequent exemplary embodiments, According to the invention, the training parameter duration of a hypoxia interval 100 is adapted (FIG. 3b) in such a way that at least 1% and at most 10% is above or below the value of the training parameter minimum oxygen content of a breathing air mixture 10 provided during the hypoxia training, specifically in such a way that oxygen saturation of blood 20 (FIG. 3a) is constantly above safety limit 30.

The section of the diagram in FIG. 3b furthermore shows the training parameters determined from the results of preliminary examinations or input parameters, length of the hypoxia interval and oxygen content in the breathing gas. When measuring the heart rate variability at rest, an RMSSD of 38 ms, a MitoOx at 1.5*10\hat{ }8 and a BHI value of 1.9 were measured. Furthermore, the breath hold test showed a time of 41 s.

Minimum target blood oxygen saturation SpO2 70, determined solely from the measurement of the heart rate variability, is 82.5%. Range of oxygen saturation SpO2 in blood 72 has a minimum value 71 of 83.5% and a maximum value 73 of 88.5%. Maximum length of the hypoxia interval 80, determined solely from the measurement of the heart rate variability, corresponds to 312 s. The range of the length of the hypoxia interval 82 has a maximum length 81 of 308 s and a minimum length 83 of 270 s. The oxygen content in breathing gas 90, determined solely from the measurement of the heart rate variability, is 12.25%. The range oxygen content in breathing gas 92 has a minimum value 91 of 12.4% and a maximum value 93 of 13.5%. The training parameters automatically determined from all the measured values are SpO2=84% for the target value of oxygen saturation in blood 74, 300 s for the length of hypoxia interval 84, and 12.5% for the oxygen content in the breathing gas. The training is then executed with these training parameters. The training parameters were all selected so that they lie in a range between 1% and 25% above or below those from the measured values of the preliminary examinations and/or the input parameters. In this case, all automatically determined training parameters 74, 84, 94 are towards more relaxed training conditions above or below training parameters 70, 80, 90 determined from the measured values of the preliminary examinations and/or input parameters.

In further embodiments, the automatically determined training parameters are at least 1.5% and at most 8%, at least 2% and at most 7% above or below the value of the training parameter of the training parameters determined from one of the measured values and/or the input parameters. Further tolerances are conceivable, depending on the acquired measured values of the user, e.g., at least 3% and at most 7.5%, at least 4% and at most 7%. In this exemplary embodiment, the tolerance is at least 2% and at most 25%.

In this way, it is possible to create training programs automatically without putting the users at risk of a lack of oxygen supply, but nevertheless to execute the training in a range which is promising for the training success. Without this method, an automatic creation of a training program would not be possible without risk for a large number of users.

Safety limit 30 is determined and set individually for each user. In this exemplary embodiment, oxygen saturation of blood 20 minus 2 percent is used for setting. When training intensity is correctly set, oxygen saturation of blood 20 remains 2 to 3% above safety limit 30 during hypoxia phase 100.

An exemplary embodiment of method 400 according to the invention for the automated execution of a hypoxia training is shown in FIG. 4. In a first method step, a plurality of measured values of a user are acquired 410 by means of an initial test and a plurality of parameters of a user are input 420 into a first computer unit.

The training parameters are determined 430 from these acquired measured values and input parameters. The training parameters comprise the duration of a hypoxia interval 100, the number of cycles of the hypoxia intervals 100 and the minimum oxygen content of a breathing air mixture 10 provided during the hypoxia training. Hypoxia phase 100 is individually adjusted via the setting options of duration 100, frequency, oxygen reduction of breathing gas 10, and oxygen saturation in blood 20.

The training parameters duration of a hypoxia interval 100, number of cycles of hypoxia intervals 100 and minimum oxygen content of a breathing air mixture 10 provided during the hypoxia training are tested 440 in a further method step. The user wears a pulse oximeter during the entire interval hypoxia training 1. The pulse oximeter is connected directly to the hypoxia device used to execute hypoxia training 1. The hypoxia device has the second computer unit with a memory or is connected to such a unit. During test 440, the training parameters duration of a hypoxia interval 100, number of cycles of the hypoxia intervals 100 and minimum oxygen content of a breathing air mixture 10 provided during the hypoxia training are compared with the values measured in real time for oxygen concentration in breathing gas 10, oxygen saturation of blood 20, heart rate 40 and safety limit 30 of oxygen saturation of blood 20.

If the determined training parameters are within the range anticipated during the initial test, and safety limit 30 of oxygen saturation of blood 20 is not undershot, a training plan is created 450 in following method step 450 and the training plan is started 460 with the determined training parameters. If the determined training parameters are not within the range anticipated during the initial test, and/or safety limit 30 of oxygen saturation of blood is not reached, the training parameters are changed 470 in such a way that the measured values for oxygen concentration in breathing gas 10, oxygen saturation of blood and heart rate 40 are again in the anticipated range and safety limit 30 of oxygen saturation of blood 20 is exceeded again. A new training plan with the changed training parameters is then created 480 and started 490 with the changed training parameters. By checking the training parameters and, if necessary, changing the training parameters in real time, safe and at the same time effective hypoxia training is achieved.

### LIST OF REFERENCE NUMERALS

1 training program/training plan
10 oxygen concentration in the breathing gas
20 oxygen saturation of blood
30 safety limit of the oxygen saturation of the blood
40 heart rate
50 hyperoxia range of the oxygen saturation in the blood
80 maximum length of the hypoxia interval determined from a measured value or input parameter
81 maximum length of the hypoxia interval
82 range of length of the hypoxia interval
83 minimum length of the hypoxia interval
84 length of the hypoxia interval in the automatically determined and executed training program
90 oxygen content of the breathing gas determined from a measured value or input parameter
91 lower limit value of the oxygen content of the breathing gas
92 range of the oxygen content of the breathing gas
93 upper limit value of the oxygen content of the breathing gas
94 oxygen content of the breathing gas in the automatically determined and executed training program
100 hypoxia interval
200 normoxia interval
300 hyperoxia interval
400 method for the automated execution of hypoxia training
410 acquisition of measured value
420 input of parameters
430 determination of training parameters
440 testing of training parameter
450 creation of training plan
460 start of training plan
470 change of training parameters
480 creation of training plan with changed parameters
490 start of training plan with changed parameters

1. A method (400) for automated execution of a hypoxia training with the method steps of
acquiring a first measured value (410) or inputting a first parameter (420)
acquiring a second measured value (410) or inputting a second parameter (420)
determining (430) training parameters from the first acquired measured value and/or the first input parameter and the second acquired measured value and/or the second input parameter,
creating (450) a hypoxia training program (1) and/or executing (460) a hypoxia training with the determined training parameters,
wherein the training parameters comprise one or more parameters from the group of duration of a hypoxia interval (100), number of cycles of the hypoxia intervals and minimum oxygen content of a breathing air mixture (10) provided during the hypoxia training,
wherein at least one value of one of the two training parameters is at least 1% and at most 25% above or below the value of that training parameter which could be determined solely from the first acquired measured value and/or the first input parameter, or solely from the second acquired measured value and/or the second input parameter.

2. The method (400) for automated execution of a hypoxia training according to claim 1,
characterized in that
the value of the one training parameter is at least 1.5% and at most 8% above or below the value of the training parameter.

3. The method (400) for automated execution of a hypoxia training according to claim 1,
characterized in that
the value of the one training parameter is at least 2% and at most 7% above or below the value of the training parameter.

4. The method (400) for automated execution of a hypoxia training according to claim 1
characterized in that
the value of the one training parameter is determined independently of the results of the measured values and/or input parameters.

5. The method (400) for automated execution of a hypoxia training according to claim 1
characterized in that
the at least one training parameter is the duration of a hypoxia interval (100) and/or the minimum oxygen content of a breathing air mixture (10) provided during the hypoxia training.

6. The method (400) for automated execution of a hypoxia training according to claim 1
characterized in that
the hypoxia training program (1) is created automatically (450, 480).

7. The method (400) for automated execution of a hypoxia training according to claim 1
characterized in that
the hypoxia training program (1) is executed automatically (460, 490).

8. The method (400) for automated execution of a hypoxia training according to claim 1
characterized in that
the measured values and/or input parameters comprise the SI value of a heart rate variability measurement, the time of a breath hold test, the results of a MinOx test, the results of an MDA-LDL test, the dose determination according to Prof. Apanasenko, the results from tests for basal respiration, ATP production, proton leak, maximum respiration, reserve capacity and/or non-mitochondrial respiration.

9. The method (400) for automated execution of a hypoxia training according to claim 1
characterized in that
the created training program (1) is created on a first computer unit (450, 480).

10. The method (400) for automated execution of a hypoxia training according to claim 1
characterized in that
the created training program (1) is transferred from a first computer unit to a second computer unit.

11. The method (400) for automated execution of a hypoxia training according to claim 1
characterized in that
the second computer unit includes a controller for executing (460, 490) the training program.

12. The method (400) for automated execution of a hypoxia training according to claim 1
characterized in that
the training parameters comprise the number of hypoxia cycles, the number of trainings of the course of treatment and/or the regeneration time between the individual trainings.

13. The method (400) for automated execution of a hypoxia training according to claim 1
characterized in that
the method (400) is executed in a personalized manner.

14. The method (400) for automated execution of a hypoxia training according to claim 1
characterized in that
the training program (1) is discontinued as a function of combinations of two values from the acquired measured values and/or input parameters and/or as a function of a single value of the acquired measured values and/or input parameters.

15. The method (400) for automated execution of a hypoxia training according to claim 1
characterized in that
the acquired measured values and/or input parameters which lead to the discontinuation of the execution of a hypoxia training and/or the creation of the hypoxia training program comprise the SI value of a heart rate variability measurement, the time of a breath hold test, the results of a MinOx test, the dose determination according to Prof. Apanasenko, the results from tests for basal respiration, ATP production, proton leak, maximum respiration, reserve capacity and/or non-mitochondrial respiration.
